(19) United States
(12) Patent Application Publication
(10) Pub. No.: US 2024/0185974 A1
(43) Pub. Date: Jun. 6, 2024

(54) SYSTEMS AND METHOD FOR ELECTRONIC EVALUATION OF RESPONDERS AND NON-RESPONDERS FOR ONE OR MORE DRUGS

(71) Applicant: Innoplexus AG, Eschborn (DE)

(72) Inventors: Irfan Tamboli, Pune (IN); Om Sharma, Pimpri-Chinchwad (IN); Harshit Gupta, Agra (IN)

(73) Assignee: Innoplexus AG, Eschborn (DE)

(21) Appl. No.: 18/060,622

(22) Filed: Dec. 1, 2022

(51) Int. Cl. G16H 20/10; G06N 20/20

(52) U.S. Cl. CPC G16H 20/10; G06N 20/20

(57) ABSTRACT

A system for an electronic identification of responders and non-responders for one or more drugs, includes a processor to obtain a pre-labeled training dataset from a first database, which includes a first type of input of labeled pretreatment data and a second type of input of labeled post-treatment response data of the plurality of subjects. The processor is configured to pre-process the pre-labeled training data to generate a modified labeled training dataset and train an ensemble machine learning (ML) model from the modified labeled training dataset. The processor is further configured to train a prediction model based on set of features extracted via the ensemble ML model to obtain a trained prediction model to detect whether an unknown subject is a responder or a non-responder for a given drug from the one or more drugs, such as only responders can be given a drug to spare non-responders from unwarranted effects.

### TECHNICAL FIELD

The present disclosure relates generally to the field of development of a predictive biomarker for screening subjects in terms of responders and non-responders for one or more drugs and more specifically, to systems and a method for electronic evaluation of responders and non-responders for one or more drugs.

### BACKGROUND

A predictive biomarker is generally used to identify individuals that are more likely to respond to exposure to a particular medical product, such as a drug. Such response could be a symptomatic benefit, improved survival, or an adverse effect. Conventionally, the process of determining the predictive biomarkers in the medical technology and bioinformatics industry depends on identification of genetic or genomic (e.g., expression levels) features, mostly in a single gene or gene product, where variants can be easily determined and associated with a positive or negative response. However, such an approach has proved to be successful in discovering drugs with only limited applications, such as only for prominent and pre-known genetic or genomics signatures that enhances the disease risk. For example, an increased expression of estrogen receptor (ER) in breast cancer serves as a predictive biomarker for the treatment with a known drug, such as tamoxifen. In other words, identification of such a predictive biomarker is straightforward when alterations, such as disease associated mutation or change in expression is known and is targeted by a given drug. However, an understanding of drug mechanisms is not clear when multiple processes and genes contribute to drug response. This causes existing systems and methods to perform erroneously, generate false positives, or simply fail to identify any definite biomarkers.

Currently, certain attempts have been made for screening subjects in terms of responders and non-responders for drugs, but all such conventional methods manifest false positives for a disease associated with complex multiple biological processes and genes. Moreover, existing systems and method to identify predictive biomarkers rely on “one size fits all” type approach where one system or model is used for various diseases, thereby causing high false positives, which is not desirable.

Therefore, in light of the foregoing discussion, there exists a need to overcome the aforementioned drawbacks associated with the conventional systems and methods associated with development of the predictive biomarkers for screening of subjects.

### SUMMARY

The present disclosure provides a system and a method for an electronic evaluation of responders and non-responders for one or more drugs. The present disclosure provides a system for an electronic detection of responders and non-responders for one or more drugs. The present disclosure provides a solution to the existing problem of how to determine which patient (i.e., an unknown or a new patient) will benefit or not from a specific drug for a specific disease without even treating the patient with the corresponding drug. In other words, existing systems and methods have a technical problem of generation of false positives when screening different unknown patients, or simply fail to identify any definite biomarkers. An aim of the present disclosure is to provide a solution that overcomes at least partially the problems encountered in the prior art and provide improved systems and an improved method for an accurate electronic evaluation as well as identification or discrimination of responders and non-responders for one or more drugs.

One or more objectives of the present disclosure is achieved by the solutions provided in the enclosed independent claims. Advantageous implementations of the present disclosure are further defined in the dependent claims.

In one aspect, the present disclosure provides a system for an electronic evaluation of responders and non-responders for one or more drugs. The system includes a processor that is configured to obtain a pre-labeled training dataset from a first database, wherein the pre-labeled training dataset comprises a first type of input of labeled pretreatment data of a plurality of subjects and a second type of input of labeled post-treatment response data of the plurality of subjects for the one or more drugs. The processor is further configured to pre-process the pre-labeled training data by applying a normalization operation and a filtering operation to generate a modified labeled training dataset, wherein the modified labeled training dataset comprises a first set of biomarkers indicative of candidate biomarkers associated with drug response for the one or more drugs. The processor is further configured to train an ensemble machine learning (ML) model from the modified labeled training dataset, wherein training the ensemble ML model comprises extracting, from the modified labeled training dataset, a set of features that comprises a second set of biomarkers indicative of prioritized biomarkers, and wherein the ensemble ML model is configured to combine output from a regression model, a classification model, and a network-based prioritization model for the extraction of the set of features. The processor is further configured to train a prediction model based on the set of features extracted via the ensemble ML model to obtain a trained prediction model, wherein the trained prediction model is used to detect whether an unknown subject is a responder or a non-responder for a given drug from the one or more drugs even before administration of the given drug to the subject when an unlabeled dataset of the unknown subject is fed to the trained prediction model.

The system enables an accurate electronic evaluation of responders and non-responders for one or more drugs. The system is able to detect responders and non-responders for a drug that involves a drug response contributed by multiple processes and multiple genes unlike the conventional systems, thereby improving accuracy and almost negligible false positives. Moreover, an output of the regression model, the classification model, and the network-based prioritization model is used for the extraction of the set of features, such as to extract the second set of biomarkers indicative of prioritized biomarkers. In other words, a combination of the regression model, the classification model, and the network-based prioritization model are beneficial to train the ensemble ML model with reduced noise and improved reliability. Furthermore, the ensemble ML model is used to train the prediction model based on the extracted set of features to obtain the trained prediction model. Therefore, the trained prediction model can be generated for a given disease and a drug combination for which the pre-labeled training dataset is available. Moreover, the trained prediction model is used to detect whether the unknown subject is the responder or the non-responder for the given drug, such that only the responders can be given the given drug to spare the non-responders from the unwarranted effects of the specified drug. In other words, the system can be used as a tool to predict the responders and the non-responder for a specific drug using patient expression profile, with improved accuracy.

In another aspect, the present disclosure further provides a system for an electronic detection of responders and non-responders one or more drugs, comprising a processor configured to obtain an unlabeled dataset of an unknown subject from a second database, pre-process the unlabeled dataset by applying a normalization operation to generate a modified unlabeled dataset, extract one or more unlabeled features from the modified unlabeled dataset by executing a pre-trained ensemble machine learning (ML) model on the modified unlabeled dataset, wherein the pre-trained ensemble ML model is configured to combine output from a regression model, a classification model, and a network-based prioritization model to extract the one or more unlabeled features. The processor is further configured to detect whether the unknown subject is a responder or a non-responder for a given drug even before administration of the given drug to the unknown subject when the extracted one or more unlabeled features of the unknown subject is fed to a pre-trained prediction model, wherein the detection of the responder or the non-responder for the given drug comprises a concurrent screening of a group of biomarkers that collectively contribute to a drug response using the pre-trained prediction model.

The system is used as a tool for an electronic detection of responders and non-responders for one or more drugs biomarkers, which is not limited to specific disease, such as the pre-trained prediction model can be pre-trained even for any disease for which the pre-labeled training dataset is available. Moreover, an output of the regression model, the classification model, and the network-based prioritization model is used by the pre-trained ensemble ML model for the extraction of the set of unlabeled features with reduced noise. Furthermore, the pre-prediction model is used to detect whether the unknown subject is the responder or the non-responder for the given drug, such as only the responders can be given the drug to spare the non-responders from the unwarranted effects of the specified drug. In other words, the system can be used as a tool to predict the responders and the non-responder for a disease and specific drug using patient expression profile, with improved accuracy, even before administration of the given drug to the unknown subject.

In yet another aspect, the present disclosure further provides a method for an electronic evaluation of responders and non-responders for one or more drugs, comprising obtaining, by a processor, a pre-labeled training dataset from a first database, wherein the pre-labeled training dataset comprises a first type of input of labeled pretreatment data of a plurality of subjects and a second type of input of labeled post-treatment response data of the plurality of subjects for the one or more drugs. The method further comprises pre-processing, by the processor, the pre-labeled training data by applying a normalization operation and a filtering operation to generate a modified labeled training dataset, wherein the modified labeled training dataset comprises a first set of biomarkers indicative of candidate biomarkers associated with drug response for the one or more drugs. The method further comprises, training, by the processor, an ensemble machine learning (ML) model from the modified labeled training dataset, wherein the training of the ensemble ML model comprises extracting, from the modified labeled training dataset, a set of features that comprises a second set of biomarkers indicative of prioritized biomarkers, and wherein the training of the ensemble ML model further comprises combining output from a regression model, a classification model, and a network-based prioritization model for the extraction of the set of features. The method further comprises, training, by the processor, a prediction model based on the set of features extracted via the ensemble ML model to obtain a trained prediction model. Moreover, the trained prediction model is used to detect whether an unknown subject is a responder or a non-responder for a given drug from the one or more drugs even before administration of the given drug to the unknown subject when an unlabeled dataset of the unknown subject is fed to the trained prediction model.

The method achieves all the advantages and technical effects of the system of the present disclosure.

It is to be appreciated that all the aforementioned implementation forms can be combined. It has to be noted that all devices, elements, circuitry, units and means described in the present application could be implemented in the software or hardware elements or any kind of combination thereof. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity which performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1A is a block diagram of a system for an electronic evaluation of responders and non-responders for one or more drugs, in accordance with an embodiment of the present disclosure;

FIG. 1B is a block diagram of a system for an electronic detection of responders and non-responders for one or more drugs, in accordance with an embodiment of the present disclosure;

FIG. 2 depicts a flowchart for an electronic evaluation of responders and non-responders for one or more drugs, in accordance with an embodiment of the present disclosure;

FIG. 3A is a diagram for identifying feature genes of a drug to obtain a score using a regression model, in accordance with an embodiment of the present disclosure;

FIG. 3B is a diagram for identifying feature genes of a drug to obtain a score using a classification model, in accordance with an embodiment of the present disclosure;

FIG. 4 is a diagram that represents a dendrogram to segregates responders and non-responders for a given drug, in accordance with an embodiment of the present disclosure;

FIG. 5 is a flowchart of a method for an electronic evaluation of responders and non-responders for one or more drugs, in accordance with an embodiment of the present disclosure; and

FIG. 6 is another flowchart of a method for an electronic evaluation of responders and non-responders for one or more drugs, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

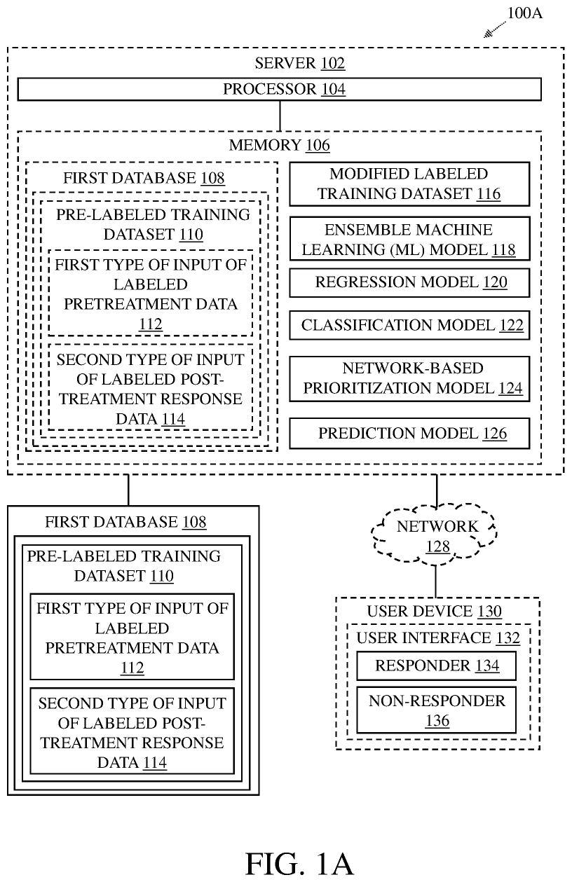

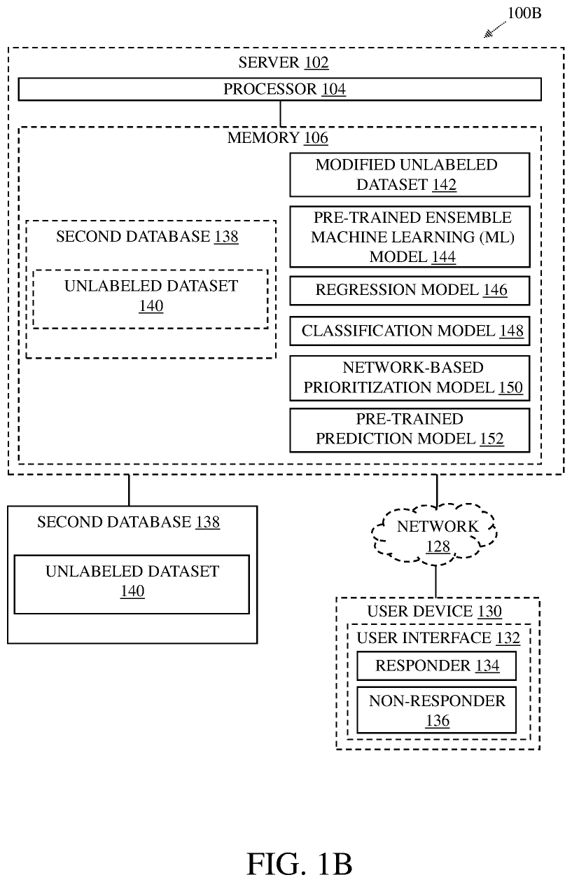

FIG. 1A is a block diagram of a system for an electronic evaluation of responders and non-responders for one or more drugs, in accordance with an embodiment of the present disclosure. With reference to FIG. 1A, there is shown a block diagram of a system 100A that includes a server 102, a processor 104 and a memory 106. The memory 106 further includes a first database 108.

The system 100A is used for an electronic evaluation of responders and non-responders for one or more drugs. The system 100A includes the server 102 that further includes the processor 104. Examples of implementation of the server 102 include, but are not limited to, a storage server, a cloud-based server, a web server, an application server, or a combination thereof.

The processor 104 refers to a computational element that is operable to respond to and processes instructions that drive the system 100A. The processor 104 may refer to one or more individual processors, processing devices and various elements associated with a processing device that may be shared by other processing devices. Additionally, the one or more individual processors, processing devices and elements are arranged in various architectures for responding to and processing the instructions that drive the system 100A. In an implementation, the processor 104 may be an independent unit and may be located outside server 102 of the system 100A. Examples of the processor 104 may include but are not limited to, a hardware processor, a digital signal processor (DSP), a microprocessor, a microcontroller, a complex instruction set computing (CISC) processor, an application-specific integrated circuit (ASIC) processor, a reduced instruction set (RISC) processor, a very long instruction word (VLIW) processor, a state machine, a data processing unit, a graphics processing unit (GPU), and other processors or control circuitry.

The memory 106 refers to a volatile or persistent medium, such as an electrical circuit, magnetic disk, virtual memory, or optical disk, in which a computer can store data or software for any duration. Optionally, the memory 106 is a non-volatile mass storage, such as a physical storage media. The memory 106 is configured to store the first database 108 that includes a pre-labeled training dataset 100. Furthermore, a single memory may encompass and, in a scenario, and the system 100A is distributed, the processor 104, the memory 106 and/or storage capability may be distributed as well. Examples of implementation of the memory 106 may include, but are not limited to, an Electrically Erasable Programmable Read-Only Memory (EEPROM), Dynamic Random-Access Memory (DRAM), Random Access Memory (RAM), Read-Only Memory (ROM), Hard Disk Drive (HDD), Flash memory, a Secure Digital (SD) card, Solid-State Drive (SSD), and/or CPU cache memory.

In operation, the processor 104 is configured to obtain the pre-labeled training dataset 110 from the first database 108. In an implementation, the first database 108 is stored in the memory 106 of the server 102. In another implementation, the first database 108 is stored outside the memory 106, as shown in FIG. 1A. In an example, the first database 108 includes one or more dataset models, such as a patient-derived xenograft (PDX) mice dataset or mouse models of human cancer database consisting of drug response datasets corresponding to each of the drugs thereof, and the like. Moreover, a specific dataset is used for a specific implementation to run the system 100A. In addition, the pre-labeled training dataset 110 may also be referred to as response datasets, training data, labelled training data, drug-treatment response data with pre-treatment expression datasets, which are stored in the first database 108. In addition, the pre-labeled training dataset 110 includes a first type of input of labeled pretreatment data 112 of a plurality of subjects. In an example, the first type of input of the labeled pretreatment data 112 corresponds to a genomics expression data of the plurality of subjects. For example, if each subject from the plurality of subjects is suffering from any disease, then the first type of input of the labeled pretreatment data 112 is taken and stored in the first database 108. For example, expression datasets of each subject are taken and stored in the first database 108 before giving any drug (or treatment) to each subject from the plurality of subjects.

In an implementation, the processor 104 is configured to receive the first type of input of the labeled pretreatment data 112 of the one or more drugs. Moreover, the first type of input of the labeled pretreatment data 112 corresponds to a form of information associated with the one or more drugs, which may be provided as a name, a pre-treatment expression data to be received by the processor 104. Furthermore, the processor 104 is configured to indicate the system 100A about the subjects (or patients) for which the mechanistic insights into the action of the one or more drugs are to be gained. In an example, the first type of input of the labeled pretreatment data 112 includes a long list of genes along with the sample expression values of each subject from the plurality of subjects. However, the first type of input of the labeled pretreatment data 112 may include some raw values, which generally are the read counts consisting of raw values from the experiment samples that require a set of data pre-processing steps to be handled by the system 100A. Additionally, the first type of input of the labeled pretreatment data 112 of the drug may be in the form of a database identification (ID). In an example, the database ID corresponds to a unique identifier of each experiment data uploaded on the first database 108, which can be handled by downloading the expression values using a database application programming interface (API).

The pre-labeled training dataset 110 further includes a second type of input of labeled post-treatment response data 114 of the plurality of subjects for the one or more drugs. In other words, upon receiving the first type of input of the labeled pretreatment data 112 of a disease, the processor 104 receives the second type of input of labeled post-treatment response data 114 related to one or more drugs of interest for which the plurality of subjects is to be screened to further gain mechanistic insights into the action of the one or more drugs. For example, if at least one subject from the plurality of subjects is suffering from tumor related disease, then corresponding subjects are given a treatment with the one or more drugs, and then the second type of input of labeled post-treatment response data 114 is obtained. In other words, the second type of input of labeled post-treatment response data 114, such as response expression datasets of corresponding subjects is taken from the plurality of subjects that are treated with the one or more drugs. Therefore, the second type of input of labeled post-treatment response data 114 corresponds to a post-treatment expression data, or a drug response data related to at least one drug response for tumor volume change datasets of the plurality of subjects. Moreover, the drug response data corresponds to a response and a non-response data based on the samples given by the plurality of subjects in an expression data, for example related to tumor volume change datasets percentage or actual tumor volume datasets. Throughout the present disclosure, the tumor volume change datasets are referred to as a set of observable characteristics related to the plurality of subjects that are treated with a dose of a specific drug from the one or more drugs. In an implementation, the second type of input of the labeled post-treatment response data 114 includes a tabular data consisting of post-treatment expression data related to the significance of responders or non-responders' subjects for the one or more drugs. For example, if a subject from the plurality of subjects shows a positive response to the dose of a specific drug from the one or more drugs or one or more given therapies, then the corresponding subject is referred to as the responder subject. However, if a subject from the plurality of subjects does not respond to the corresponding dose of a specific drug from the one or more drugs or one or more given therapies, then the corresponding subject is referred to as the non-responder subject. Therefore, the second type of input of the labeled post-treatment response data 114 includes data related to a list of response samples and non-response samples of the plurality of subjects based on the significance of responders or non-responders' subjects for the one or more drugs.

The processor 104 is further configured to pre-process the pre-labeled training dataset 110 by applying a normalization operation and a filtering operation to generate a modified labeled training dataset 116. In an implementation, the normalization operation is used for selecting the second type of input from the pre-labeled training dataset 110 related to at least one drug from the one or more drugs from the list of response samples and non-response samples of the plurality of subjects. Herein, the list of response samples and non-response samples is pre-compiled by the processor 104 and stored in the first database 108. Thereafter, the processor 104 is configured to pre-process the pre-labeled training dataset 110 by applying the normalization operation on a threshold for classifying the responders and the non-responders as per the requirements of the system 100A. Optionally, the processor 104 is configured to apply the normalization operation on the pre-labeled training dataset 110 by merging the first type of input of the labeled pretreatment data 112 and the second type of input of the labeled post-treatment response data 114. The processor 104 is configured to normalize the pre-labeled training dataset 110, which is associated with the one or more drugs. In an example, the pre-labeled training dataset 110 (e.g., expression data) is normalized, such that the second type of input of labeled post-treatment response data 114 is converted into labels and actual tumor volume. For example, table 1. represents a feature designed to determine the correlation of gene expression data with the tumor volume change response datasets using spearman correlation.

TABLE 1

| Gene | TVC_corr | rank_TVC_corr |
|---|---|---|
| NPNT | 0.803 | 0.999 |
| TEX2 | −0.792 | 0.999 |
| SOX17 | −0.778 | 0.998 |
| HS6ST2 | 0.772 | 0.997 |
| NPAS3 | −0.763 | 0.996 |
| SIRPA | 0.753 | 0.996 |
| C6orf132 | −0.750 | 0.995 |
| CCDC6 | −0.749 | 0.994 |
| RAB11FIP4 | −0.737 | 0.994 |
| UBE4B | −0.735 | 0.993 |
| PLXNA4 | 0.733 | 0.992 |
| CYP2A13 | 0.722 | 0.991 |
| SIM2 | 0.719 | 0.991 |
| EXD1 | −0.718 | 0.990 |
| GOLM1 | 0.718 | 0.990 |
| ENKD1 | 0.716 | 0.988 |
| PTPN13 | 0.714 | 0.988 |
| AQP5 | −0.708 | 0.987 |
| PPA2 | 0.706 | 0.986 |

In an implementation, the normalization operation further includes executing a Z-score log-normalization or other standard deviation-based log-normalization to reduce data noise in the pre-labeled training dataset 110. In an example, the processor 104 is configured to execute the Z-score log-normalization on the pre-labeled training dataset 110 (e.g., expression data) to generate the modified labeled training dataset 116. In another example, the processor 104 is configured to execute the standard deviation-based log-normalization, such as proprietary algorithms on the pre-labeled training dataset 110 (e.g., expression data) to generate the modified labeled training dataset 116. Moreover, a low computational power is required to reduce the data noise in the pre-labeled training dataset 110.

The processor 104 is further configured to perform, filtering operations on the pre-labeled training dataset 110, such as for filtering genes from the pre-labeled training dataset 110 based on the responders/non-responders' significance statistically. In an implementation, the filtering operation includes segregating and filtering out biomarkers that do not correlate with the drug response for the one or more drugs of a disease in the pre-labeled training dataset 110. For example, the processor 104 is configured to analyze the drug response for the one or more drugs of the disease for a subject, such as a drug response associated with tumor volume change dataset along with the first type of input of labeled pretreatment data 112 for the same subject for which the information of responders/non-responders values is related. Furthermore, the processor 104 is configured to perform filtering operations by segregating the biomarkers that do not correlate with the drug response for the one or more drugs with the tumor volume change in the pre-labeled training dataset 110. In addition, the processor 104 is configured to perform filtering operation by filtering out the biomarkers that do not correlate with the drug response for the one or more drugs with the tumor volume change in the pre-labeled training dataset 110, which is a specific dataset of the first database 108. Such as, to filter and segregate the biomarkers for the responders and the non-responders from the plurality of subjects. As a result, the first set of biomarkers indicative of candidate biomarkers associated with the drug response for the one or more drugs is obtained after segregating and filtering out the biomarkers that do not correlate with the drug response for the one or more drugs of the disease in the pre-labeled training dataset 110. In other words, the modified labeled training dataset 116 includes the first set of biomarkers indicative of candidate biomarkers associated with drug response for the one or more drugs. Similarly, the first set of biomarkers indicative of candidate biomarkers also includes the drug response for the one or more drugs related to other diseases.

The modified labeled training dataset 116 is further used to train an ensemble machine learning (ML) model 118. In other words, the processor 104 is configured to train the ensemble ML model 118 from the modified labeled training dataset 116. In an implementation, the ensemble ML model 118 is based on a machine learning algorithm based on a computational approach, which can leverage a growing number of large-scale human genomics and proteomics datasets or any other omics datasets to make the system 100A generalized and more precise to the predictions. Further, the ensemble ML model 118 is used to prioritize targets according to their ability to contribute to the prediction of the target variable. In addition, the ensemble ML model 118 predicts the response of a subject based upon the genes that are selected. Furthermore, training the ensemble ML model 118 includes extracting, from the modified labeled training dataset 116, a set of features that includes a second set of biomarkers indicative of prioritized biomarkers. Optionally, the processor 104 is configured to use an exhaustive feature selector, and sequential feature selector for extracting the set of features from the modified labeled training dataset 116, such as the set of features that includes the second set of biomarkers indicative of prioritized biomarkers.

In an implementation, the ensemble ML model 118 is used for expressing covariance similarly to a matrix score based on the tumor volume change numerical values and the expression values for an individual gene. The score can infer the probability of a gene to be important for further analysis. In an example, the score ranges from ‘−1’ to ‘1’, such as score ‘−1’ refers to the distribution similarity in reverse order and score ‘1’ refers to the similarity to be strong in the same direction. For example, if the expression of a gene is increasing the tumor volume should increase in the same manner and vice versa.

In an implementation, the processor 104 is further configured to identify targets of the drug to obtain a drug target list. In an example, the target of the drug is corresponding to a protein, which is intrinsically associated with a particular disease process. Furthermore, the target could be addressed by the drug to produce a desired therapeutic effect. Such as, the target is identified and characterized by identifying the function of a possible therapeutic agent, and the therapeutic agent may be a gene and/or protein and corresponding role in the disease. In this regard, at least one drug interacts with multiple targets rather than with a single target. Subsequently, the targets that are identified are listed down in a target list, and the target list includes all the targets relevant to the at least one drug given as input to the processor 104.

Optionally, the processor 104 is configured to use a clustering algorithm (e.g., using a clustering model) to determine an improved silhouette score, which is used to determine a top and major cluster within the gene targets. In an example, the processor 104 is configured to use other metrics, such as Davies-Bouldin Index, Calinski-Harabasz index, and the like to exactly check the distances within the clusters and inter cluster distances. Such other metrics are used by the processor 104 to determine the top region of genes to focus on and keep them on priority in further processing steps.

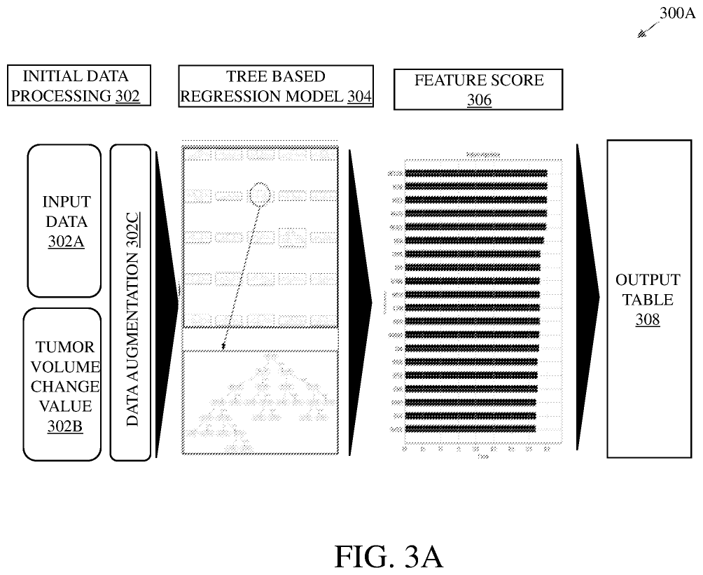

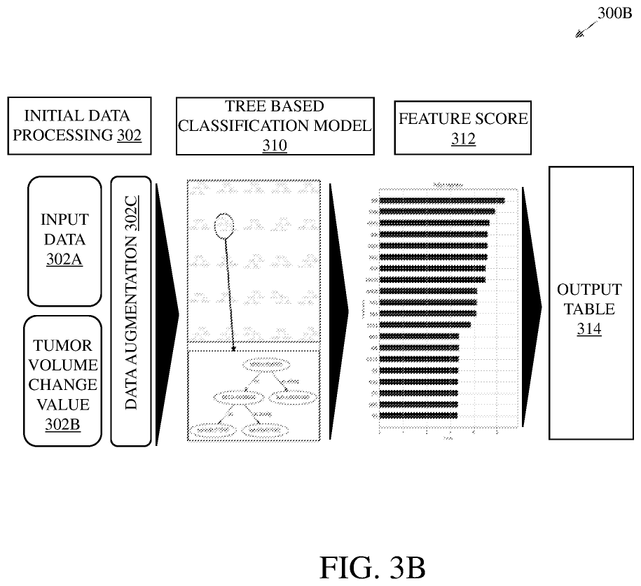

In an implementation, the processor 104 is further configured to train the ensemble machine learning (ML) model 118 to determine and enrich matrix scores of the set of features based on gene expression data along with tumor volume change data. In an example, a regression model 120 is implemented on the modified labeled training dataset 116, which includes a low number of rows (i.e., sample count) as compared to columns (i.e., number of genes). Moreover, the regression model 120 may also be referred to as a feature selection regression module to perform regression analysis on the modified labeled training dataset 116, as further shown, and described in FIG. 3A. The processor 104 is further configured to generate gene combinations by placing zeros in combinations to keep the target variables unchanged. In an example, “number_of_iterations” parameters are used to control the number of iterations or combinations of automation to generate a first score (or a result) of the enriched increased number of rows to be provided in the modified labeled training dataset 116. In an implementation, the regression model 120 is a decision-tree based regression model in which the first score is assigned to each gene in the modified labeled training dataset 116 to identify one or more feature biomarkers of the one or more drugs. In other words, the modified labeled training dataset 116 is used for identification as well as the selection of the one or more feature biomarkers to train the decision-tree based regression model, such as each feature biomarker from the second set of biomarkers later can be prioritized using the first score. In an example, the first score is used with a clustering model to label the important genes based on covariance similarity.

In an implementation, the first score is used as a training data in a feature selection regression module that includes a comparative number of rows and columns. In an example, training is performed through BayesSearchCV with scientifically selected ranges of hyperparameters. Such as, bayesian optimization over hyperparameters as a tune procedure, which takes input in ranges and output top parameters with the first score, as shown below:
num_estimators(number of Trees): Range(300, 700)
col_sample_bytree (Number of columns selected while preparing a tree): Range(5,30)
learning_rate: array ([0.001, 0.05, 0.1])
alpha(Regularization parameter): array([0.01, 0.1, 0.2])
and the rest of the parameters kept constant.

The processor 104 is further configured to design a loss function to keep the convergence at a level-wise tree-based approach of the decision-tree based regression model. In an example, the processor 104 is further configured to use a mean absolute error (MAE) and root mean squared error (RMSE) for training the ensemble ML model 118 with reduced errors. Moreover, a custom loss function is used to converge the ensemble ML model 118 efficiently and enables to enrich features (e.g., the set of features) of the first score, such as a feature score matrix. Furthermore, an output of the regression model 120 is obtained, as shown below in table 2.

TABLE 2

| Gene | Rfeatures | rank_Rfeatures |
|---|---|---|
| CYP2A13 | 0.027 | 1.000 |
| BTLA | 0.025 | 0.999 |
| PRICKLE1 | 0.025 | 0.999 |
| NLGN3 | 0.025 | 0.998 |
| CDR2L | 0.025 | 0.997 |
| IPCEF1 | 0.024 | 0.996 |
| KRT6A | 0.024 | 0.996 |
| C6orf132 | 0.024 | 0.995 |
| EDC4 | 0.024 | 0.994 |
| SLC48A1 | 0.024 | 0.994 |
| DNMT1 | 0.024 | 0.992 |
| CAMK2D | 0.023 | 0.991 |
| C19orf68 | 0.022 | 0.988 |
| CDCA7 | 0.022 | 0.988 |
| KDELR1 | 0.022 | 0.987 |
| CCNA1 | 0.021 | 0.986 |
| FAM181A | 0.021 | 0.986 |
| CDH6 | 0.021 | 0.985 |
| AMY1B | 0.020 | 0.983 |

The processor 104 is further configured to train the ensemble machine learning (ML) model 118 to determine and enrich the matrix scores of the set of features based on gene expression data along with volume change labeled, such as responders/non-responders (R/NR) data. In an example, a classification model 122 is implemented on the modified labeled training dataset 116, which includes a low number of rows (i.e., sample count) as compared to columns (i.e., number of genes). Optionally, the classification model 122 may also be referred to as a feature selection classification module, as further shown, and described in FIG. 3B. Moreover, the processor 104 is configured to generate the gene combinations for placing zeros in combinations to keep the target variables unchanged. In an example, a “number_of_iterations” parameters are used to control the number of iterations or combinations the automation should generate a second score (or a result) of the enriched and increased number of rows to be provided in the modified labeled training dataset 116. In an implementation, the classification model 122 is a gradient boosting-based classification in which a second score is assigned to each gene in the modified labeled training dataset 116 to identify one or more feature biomarkers of the one or more drugs. In other words, the modified labeled training dataset 116 is used for identification as well as selection of the one or more feature biomarkers to train the decision-tree based regression model, such as each feature biomarker from the second set of biomarkers later can be prioritized using the second score. In an example, a hyperparameter tuned for the category target variable is used to generate the second score, such as a feature weight score for each gene in the modified labeled training dataset 116. In another example, a hyperparameter tuned for numerical target variables is used to generate the second score, such as the feature weight score for each gene in the modified labeled training dataset 116.

Moreover, the second score is used as a training data in the classification model 122, which includes a comparative number of rows and columns. In an example, training is performed through BayesSearchCV with scientifically selected ranges of hyperparameters. Such as, bayesian optimization over hyperparameters as a tuning procedure, which includes input as in ranges and outputs the top parameters with the second score, as shown below:
num_estimators(number of Trees): Range(200, 600)
max_depth(the max depth a tree should lead while predicting): Range(2,6)
learning_rate(Weak learners weight assigning rate): array([0.001, 0.05, 0.1])
gamma(Minimum loss reduction required to make a further partition on a leaf node): array([0, 0.1, 0.5])
and the rest of the parameters kept constant.

The processor 104 is further configured to a loss function to keep the convergence at a level-wise tree-based approach and binary logistic log loss. In addition, an entropy score is used to minimize the error while training. In an example, a custom loss function is used to converge the algorithm efficiently and enables it to enrich features (e.g., the set of features) of the second score. Furthermore, an output of the classification model 122 is obtained, as shown below in the table 3.

TABLE 3

| Gene | Cfeatures | rank_Cfeatures |
|---|---|---|
| NPNT | 1.08E−01 | 1.000 |
| MTMR4 | 6.97E−02 | 0.999 |
| FERMT1 | 6.18E−02 | 0.999 |
| CYB5R4 | 5.92E−02 | 0.998 |
| SIRPA | 5.89E−02 | 0.997 |
| DOCK2 | 5.87E−02 | 0.996 |
| ACSL4 | 5.69E−02 | 0.996 |
| KIAA1551 | 5.61E−02 | 0.995 |
| OLFML2B | 4.46E−02 | 0.994 |
| THOC2 | 4.43E−02 | 0.994 |
| THSD4 | 4.40E−02 | 0.993 |
| CUEDC1 | 3.28E−02 | 0.992 |
| CCDC138 | 5.04E−03 | 0.991 |
| AGPS | 5.03E−03 | 0.991 |
| ABHD3 | 5.03E−03 | 0.990 |
| DDI2 | 4.95E−03 | 0.989 |
| MICAL1 | 4.88E−03 | 0.988 |
| ETV5 | 4.85E−03 | 0.988 |
| DNMT1 | 4.82E−03 | 0.987 |

In an implementation, the network-based prioritization model 124 is a gene network-based gene prioritization model in which a cumulative score is computed based on the first score and the second score of a corresponding gene to generate the set of features that includes the second set of biomarkers indicative of the prioritized biomarkers. For example, the processor 104 is configured to use the network-based prioritization model 124 through a network gene prioritization algorithm based on signalling pathway impact analysis (SPIA) methodologies to generate the cumulative score, which may also be referred to as a cumulative confidence score, a gene-perturbation score, or a gene-based impact score incorporating features of the first score and the second score. In addition, the SPIA methodologies incorporate all the pathways related to the first database 108 (e.g., kegg database resource) and enrich the cumulative score, such as from the network-based prioritization model 124. In addition, the cumulative score is used by the processor 104 to determine how much the corresponding gene in the modified labeled training dataset 116 is beneficial for the system 100A of genes across pathways.

In an implementation, the processor 104 is further configured to enrich target-based pathways using curated databases from different sources for all the significant genes in a frame. For example, enriching pathways using proprietary curated databases for highly relevant genes in the modified labeled training dataset 116. In addition, the cumulative score demonstrates the perturbation score of a gene based on relevant pathways.

In an example, all edge weights emerging from that specific gene and coming in edges to leverage the weights are interpreted and incorporated to find out the cumulative score with the help of network hierarchy. Moreover, such methodology incorporates the edge weight of gene interactions. An exemplary scenario of a network perturbed cumulative score for network-based prioritization model 124 based on genes and pathways enriched through the gene list is shown below in table 4.

TABLE 4

| Gene | network_score | rank_network_score |
|---|---|---|
| RAPGEF3 | 0.073 | 0.999 |
| RASSF5 | 0.026 | 0.999 |
| FGFR2 | 0.023 | 0.998 |
| SOX9 | 0.020 | 0.997 |
| KCNJ8 | 0.013 | 0.996 |
| HCN4 | 0.013 | 0.996 |
| RIN1 | 0.011 | 0.995 |
| BMP2 | 0.009 | 0.994 |
| BMP4 | 0.007 | 0.994 |
| GRIA3 | 0.007 | 0.993 |
| DIAPH2 | 0.006 | 0.992 |
| RALBP1 | 0.005 | 0.991 |
| ACOX1 | 0.005 | 0.991 |
| CNGB1 | 0.004 | 0.990 |
| GNAI1 | 0.004 | 0.989 |
| ADCY5 | 0.003 | 0.988 |
| RPS6KB1 | 0.003 | 0.988 |
| ADCY9 | 0.003 | 0.987 |
| ERBB4 | 0.003 | 0.986 |

The ensemble ML model 118 is further configured to combine an output from the regression model 120, the classification model 122, and the network-based prioritization model 124 for the extraction of the set of features. In other words, training the ensemble ML model 118 further includes, combing the output from the regression model 120, the classification model 122, and the network-based prioritization model 124 for the extraction of the set of features. For example, the processor 104 is configured to combine the first score, the score, and the cumulative score for training the ensemble ML model 118 model from the modified labeled training dataset 116 and to extract the set of features that includes the second set of biomarkers indicative of prioritized biomarkers. As a result, the ensemble ML model 118 is exhaustive, precise, and based on the modified labeled training dataset 116, such as based on expression data and response data labels to prioritize the genes, which can be a potential candidate for the first set of biomarkers indicative of predictive biomarkers.

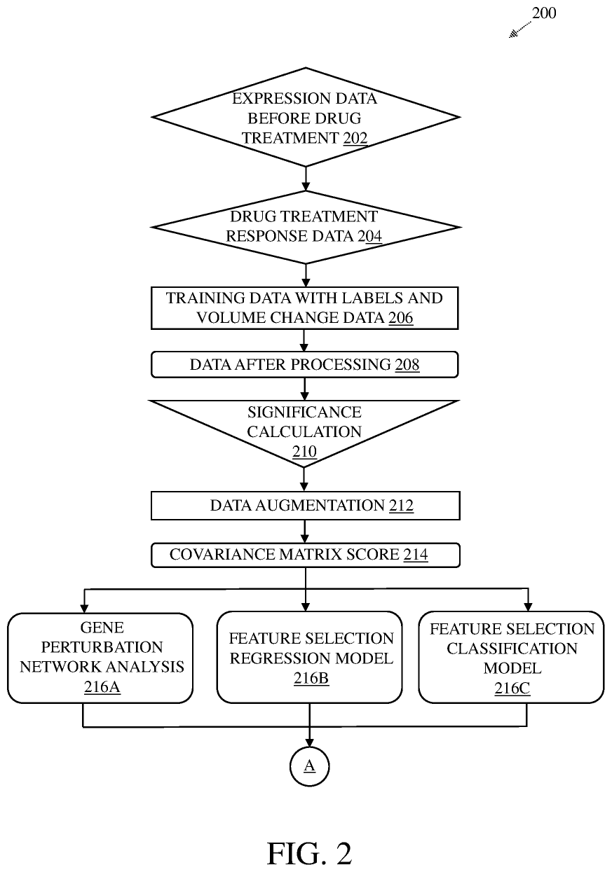

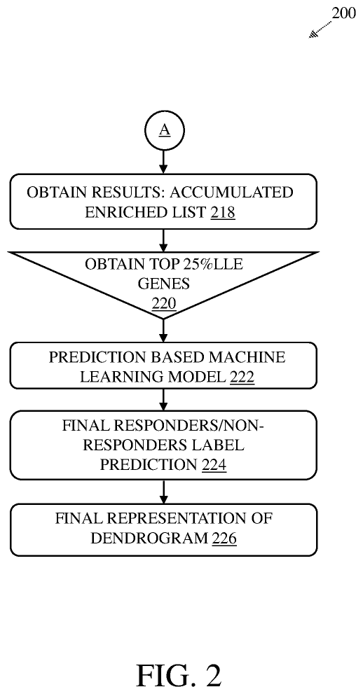

In an implementation, the processor 104 is configured to compare output from the regression model 120, the classification model 122, and the network-based prioritization model 124 to calculate an accumulated score, which is used to generate a final prioritized gene list for the system 100A. The processor 104 is used to reduce the data size and reduces the computational power from a large data corpus to a small number of genes in a final corpus, such as in the accumulated score. In an example, the processor 104 is configured to determine the number of features, which are designed with scientifically picked hyper-parameters and proprietary algorithms. In addition, a combined output of the regression model 120, the classification model 122, and the network-based prioritization model 124 is used to prioritize the genes, which can be used to train the prediction model 126 for identifying a predictive biomarker for a selection of treatment for a specific patient population, as further shown, and described in FIG. 2. In an example, the processor 104 is configured to use of any type of cancer model (e.g., mice tumor xenograft, tumor cell line and the like) to generate training dataset and to build prediction models, such as to train the prediction model 126, which can be used to predict the response in patients. In an example, the processor 104 is configured to combine the output of the regression model 120, the classification model 122, and the network-based prioritization model 124, such as to emphasize the accumulated score based on artificial intelligence. Such as, the accumulated score is on high priority, as shown below with a formula as well as in table 5.

Score_Combined_1=Mean(Score_Network,Score_Corr)

Score_Combined_2=Mean(Score_Regr,Score_Classify)

Net_Score=WHM(Score_(Combined_1)+Score_(Combined_2))

Where, WHM corresponds to weighted harmonic mean.

TABLE 5

| | | | Confi- |
|---|---|---|---|
| Gene | Score_Combined_1 | Score_Combined_2 | dence |
| ACSL4 | 0.920 | 0.962 | 0.941 |
| CAMK2D | 0.979 | 0.724 | 0.832 |
| PRICKLE1 | 0.959 | 0.727 | 0.827 |
| ACOX1 | 0.961 | 0.718 | 0.822 |
| ANTXR1 | 0.708 | 0.956 | 0.813 |
| CCNA1 | 0.930 | 0.721 | 0.812 |
| ATP2B2 | 0.907 | 0.715 | 0.800 |
| BTLA | 0.671 | 0.972 | 0.794 |
| AKAP2 | 0.660 | 0.947 | 0.778 |
| ARID1A | 0.644 | 0.956 | 0.769 |
| IL22RA1 | 0.833 | 0.708 | 0.765 |
| BMP4 | 0.826 | 0.706 | 0.761 |
| ABHD3 | 0.622 | 0.960 | 0.755 |
| DNMT1 | 0.606 | 0.990 | 0.752 |
| BMPR1B | 0.793 | 0.703 | 0.745 |
| CBLB | 0.803 | 0.694 | 0.745 |
| ADAM22 | 0.604 | 0.962 | 0.742 |
| C1orf105 | 0.602 | 0.954 | 0.738 |
| ADAM29 | 0.600 | 0.948 | 0.735 |
| NPNT | 0.733 | 0.731 | 0.732 |
| SIRPA | 0.731 | 0.730 | 0.730 |
| CYP2A13 | 0.729 | 0.728 | 0.728 |
| C6orf132 | 0.730 | 0.726 | 0.728 |
| APLNR | 0.745 | 0.707 | 0.726 |

The processor 104 is further configured to train the prediction model 126 based on the set of features extracted via the ensemble ML model 118 to obtain a trained prediction model. In an example, the processor 104 is configured to apply data augmentation methodologies to generalize and train the prediction model 126, such as by building a modified support vector machine (SVM) based hyperparameters tuned prediction model to predict a target class. Moreover, the processor 104 is configured to sort and select a list of top genes, which are with high scores in the modified labeled training dataset 116. In an example, manual validation is also required to determine the best possible genes throughout the treatment and indication of the one or more feature biomarkers of the one or more drugs. Thereafter, a training data is prepared with only high-score genes with the same augmentation expression data and tumor volume change and labels. In an example, a stacked model is developed to provide preciseness to the prediction model 126, such as by using SVM-based algorithms to mitigate the problem of fewer data points. The prediction model 126 is used to predict a final target class of unknown samples. Moreover, the trained prediction model is used to detect whether an unknown subject is a responder or a non-responder for a given drug from the one or more drugs even before administration of the given drug to the unknown subject when an unlabeled dataset of the unknown subject is fed to the trained prediction model. For example, if the unlabeled dataset of the unknown subject is fed to the trained prediction model, then the trained prediction model is used to determine a set of genes to segregate the samples of the responder or the non-responder. Beneficially as compared to conventional approaches, the trained prediction model can be used to predict, if the unknown subject will benefit from the given drug or not, such as with the help of an expression data for the set of genes for the unlabeled dataset of the unknown subject and even before administration of the given drug.

In an implementation, the trained prediction model is based on artificial intelligence. In an example, the trained prediction model is used for predicting labels with an improved cross validation accuracy of around 93% on such a small dataset as compared to conventional approaches. In another example, an F1-score (e.g., harmonic mean of precision (TP/(TP+FN)) and recall (TP/(TP+FP))) is used to determine granularity of the trained prediction model, such as to predict the labels of the trained prediction model. Moreover, the trained prediction model is used to predict the labels for the unknown subject, which have the expression of identified genes.

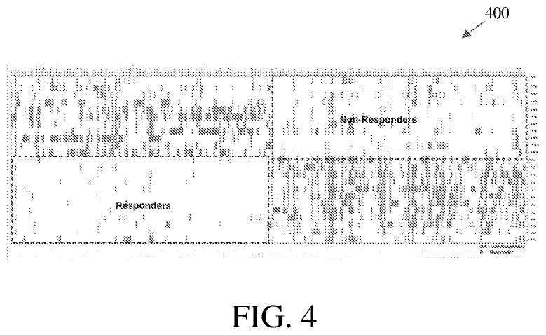

In an implementation, the processor 104 is further configured to generate and control display of a dendrogram to visually represent the unlabeled dataset of the unknown subject or other unknown subjects to distinguish the responders from the non-responders. In other words, a dendrogram is generated to segregate the responder or the non-responder for a given drug from the one or more drugs even before the administration of the given drug. The dendrogram can be prepared for getting the perfect segregation of labels and to showcase the results. Finally, the dendrogram can be prepared for getting the perfect segregation of labels and to showcase the results. An exemplary scenario of the dendrogram to represents complete segregation between the responder or the non-responder for a given drug as generated with the help of genes is shown and described in FIG. 4. In an example, a user device 130 is connected with the server 102 through a network 128. The network 128 is configured to allow dual-communicate between the system 100A and the user device 130. Examples of the network 128 include but are not limited to, a network interface card, transceiver, and the like. Similarly, the user device 130 may be a cellular device, a computer, a laptop, or any other device. Moreover, the processor 104 is configured to represent the unlabeled dataset of the unknown subject or other unknown subjects on a user interface (UI) 132 of the user device 130. Moreover, a UI element is used to represent that the subject is a responder for a given drug from the one or more drugs, such as through a responder 134 UI element. Similarly, another UI element is used to represent that the subject is a non-responder for a given drug from the one or more drugs, such as through a non-responder 136 UI element.

The system 100A enables an accurate electronic evaluation of responders and non-responders for one or more drugs. The system 100A is able to detect responders and non-responders for a drug that involves a drug response contributed by multiple processes and multiple genes unlike the conventional systems, thereby improving accuracy and almost negligible false positives. The system 100A is used as a tool for an electronic evaluation of responders and non-responders for one or more drugs, which is not limited to specific disease, such as the prediction model 126 can be trained even for any disease for which the pre-labeled training dataset is available. Moreover, an output of the regression model 120, the classification model 122, and the network-based prioritization model 124 is used for the extraction of the set of features, such as to extract the second set of biomarkers indicative of prioritized biomarkers. In other words, the regression model 120, the classification model 122, and the network-based prioritization model 124 are beneficial to train the ensemble ML model 118 with reduced noise. Furthermore, the ensemble ML model 118 is used to train the prediction model 126 based on the extracted set of features to obtain the trained prediction model. Therefore, the trained prediction model can be generated for any disease for which the pre-labeled training dataset is available. Moreover, the trained prediction model is used to detect whether the unknown subject is the responder or the non-responder for the given drug, such as only the responders can be given the given drug to spare the non-responders from the unwarranted effects of the specified drug. In other words, the system 100A can be used as a tool to predict the responders and the non-responder for a disease and specific drug using patient expression profile, with improved accuracy, even before administration of the given drug to the unknown subject. The system 100A provides an efficient way to identify predictive biomarkers for a drug selection based on a population of the unknown subjects, such as any substance that is used to prevent, diagnose, treat, or relieve symptoms of a disease or any abnormal condition. The system 100A is based on the association of the drug with samples of the unknow subjects (or patients) that are considered while gaining predictive insights. Furthermore, the system 100A reduces an overall cost as well processing time by making use of predictive biomarker identification techniques that enable the screening of multiple gene target groups simultaneously, thereby resulting in precise results for gaining mechanistic insights into the action of a drug. The system 100A is used for the identification of target subjects from the plurality of subjects into the action of drug-using predictive biomarker techniques. The system 100A is further used for the development of the trained prediction mode based on the genomics data of individual patients. The system 100A is used for identifying highly correlated genes with drug response for a given drug in curative applications against said disease.

FIG. 1B is a block diagram of a system for an electronic detection of responders and non-responders for one or more drugs, in accordance with an embodiment of the present disclosure. With reference to FIG. 1B, there is shown a block diagram of a system 100B that includes the server 102, the processor 104 and a memory 106. The memory 106 further includes a second database 138.

The system 100B is used for an electronic detection of responders and non-responders of one or more drugs. The system 100B includes the processor 104. In operation, the processor 104 is configured to obtain an unlabeled dataset 142 of an unknown subject from the second database 138. In an example, the unlabeled dataset 142 acts as a novel and experimental unlabeled data of the unknown subject, before the administration of the given drug to the unknown subject. In an implementation, the second database 138 is stored in the memory 106 of the server 102. In another implementation, the second database 138 is stored outside the memory 106, as shown in FIG. 1B. Thereafter, the processor 104 is configured to pre-process the unlabeled dataset 142 by applying a normalization operation to generate a modified unlabeled dataset 142. Such as integration of the unlabeled dataset 142 and mapping in regard to a specific number of features identified to generate the modified unlabeled dataset 142. In an implementation, the normalization operation includes executing a Z-score log-normalization or other standard deviation-based log-normalization to reduce data noise. In an example, the processor 104 is configured to execute the Z-score log-normalization on the unlabeled dataset 142 (e.g., expression data) to generate the modified unlabeled dataset 142. In another example, the processor 104 is configured to execute the standard deviation-based log-normalization, such as proprietary algorithms on the unlabeled dataset 142 to generate the modified unlabeled dataset 142.

The processor 104 is further configured to extract one or more unlabeled features from the modified unlabeled dataset by executing a pre-trained ensemble machine learning (ML) model 144 on the modified unlabeled dataset 142. The pre-trained ensemble ML model 144 is configured to combine output from a regression model 146, a classification model 148, and a network-based prioritization model 150 to extract the one or more unlabeled features. In an implementation, the regression model 146 is a decision-tree based regression model in which a first score is assigned to each gene in the modified unlabeled dataset. In another implementation, the classification model 148 is a gradient boosting-based classification in which a second score is assigned to each gene in the modified labeled training dataset. In yet another implementation, the network-based prioritization model 150 is a gene network-based gene prioritization model in which a cumulative score is computed based on the first score and the second score of a corresponding gene to generate the one or more unlabeled features.

The processor 104 is further configured to detect whether the unknown subject is a responder or a non-responder for a given drug even before administration of the given drug to the unknown subject when the extracted one or more unlabeled features of the unknown subject is fed to a pre-trained prediction model 152. Moreover, the detection of the responder or the non-responder for the given drug includes a concurrent screening of a group of biomarkers that collectively contribute to a drug response using the pre-trained prediction model 152. The pre-trained prediction model 152 is developed to predict the responder or the non-responder for a given drug. The pre-trained prediction model 152 is pre-trained using genomics data from responders and non-responders prior to drug treatment. Moreover, depending upon the response to the drug, each patient or a disease model can be labeled as the responder or the non-responder. The pre-trained prediction model 152 learns from a labeled genomics data and generates a successful discriminatory signature to distinguish between the responders and the non-responders. Such a signature is later mapped into novel or experimental unlabeled patient data so that prediction of likely responders and non-responders can be performed with reduced errors

In an implementation, the processor 104 is further configured to generate an electronic visualization and control display of the generated electronic visualization to visually represent the unlabeled dataset of the unknown subject or other unknown subjects to distinguish the responders from the non-responders.

In an example, the user device 130 is connected with the server 102 through a network 128. The network 128 is configured to allow dual-communicate between the system 100B and the user device 130. Moreover, the processor 104 is configured to generate the electronic visualization and control display of the generated electronic visualization on the user interface (UI) 132 of the user device 130. Such as, the electronic visualization is used to represent the unlabeled dataset of the unknown subject or other unknown subjects on the user interface (UI) 132 of the user device 130. Moreover, a UI element is used to represent that the subject is a responder for a given drug from the one or more drugs, such as through a responder 134 UI element. Similarly, another UI element is used to represent that the subject is a non-responder for a given drug from the one or more drugs, such as through a non-responder 136 UI element. In an example, a dendrogram visualization is used for labels of the responders or the non-Responders on identified features, such as dendrogram visualization is used to showcase the actual segregation on the responder and the non-responders' labels.

The system 100B is used as a tool for an electronic detection of responders and non-responders for one or more drugs biomarkers, which is not limited to specific disease, such as the pre-trained prediction model 152 can be pre-trained even for any disease for which the pre-labeled training dataset is available. Moreover, an output of the regression model 146, the classification model 148, and the network-based prioritization model 150 is used by the pre-trained ensemble ML model 144 for the extraction of the set of unlabeled features with reduced noise. Furthermore, the pre-prediction model 152 is used to detect whether the unknown subject is the responder or the non-responder for the given drug, such as only the responders can be given the given drug to spare the non-responders from the unwarranted effects of the specified drug. In other words, the system 100B can be used as a tool to predict the responders and the non-responder for a disease and specific drug using patient expression profile, with improved accuracy, even before administration of the given drug to the unknown subject.

FIG. 2 depicts a flowchart for an electronic evaluation of responders and non-responders for one or more drugs, in accordance with an embodiment of the present disclosure. FIG. 2 is described in conjunction with elements from FIG. 1A. With reference to FIG. 2, there is shown a flowchart 200 that includes a series of operations from 202-to-226. The processor 104 (of FIG. 1A) is configured to execute the flowchart 200.

At operation 202, the processor 104 is configured to obtain an expression data before drug treatment. In an implementation, the expression data is also referred to as a first type of input of labelled pre-treatment data of a plurality of subjects. Thereafter, at operation 204, the processor 104 is configured to obtain a drug treatment response data. In an implementation, the drug treatment response data may also refer to as a second type of input of labeled post-treatment response data of the plurality of subjects for the one or more drugs. Moreover, the expression data before drug treatment as well as the drug treatment response data are collectively referred to as a pre-labeled training data. After that, at operation 206, the processor 104 is configured to perform training of data with labels and volume change data. In other words, the processor 104 is configured to pre-process the pre-labeled training data by applying a normalization operation and a filtering operation to generate a modified labeled training dataset. Furthermore, at operation 208, a modified data after processing is obtained, such as a modified labeled training dataset including a first set of biomarkers indicative of candidate biomarkers associated with drug response for the one or more drugs is obtained. After that, at operation 210, the processor 104 is configured to perform a significance calculation to get covariance matrix score, and at operation 212, the processor 104 is configured to perform data augmentation. As a result, at operation 214, a covariance matrix score is obtained.

Furthermore, at operation 216A, the processor 104 is configured to perform gene perturbation network analysis. In addition, at operation 216B, the processor 104 is configured to obtain a feature selection regression model. Moreover, the processor 104 is further configured to obtain a feature selection-classification model, such as at operation 216C. Furthermore, at operation 218, the processor 104 is configured to obtain results that are accumulated enriched list to obtain a top 25% LLEgenes, as shown at operation 220. At operation 222, the processor 104 is configured to train a prediction-based machine learning model based on the set of features extracted via the ensemble ML model to obtain a trained prediction-based machine learning (ML) model (e.g., the pre-trained prediction model 152 of FIG. 1B). Furthermore, at operation 224, the processor 104 is configured to perform final responders or non-responders label prediction using the trained prediction-based ML model. In other words, the trained prediction-based ML model is used to detect whether an unknown subject is a responder or a non-responder for a given drug from the one or more drugs even before the administration of the given drug to the unknown subject when an unlabeled dataset of the unknown subject is fed to the trained prediction model. Finally, at operation 226, the final representation of the dendrogram is obtained, as further shown, and described in FIG. 4. In an implementation, the processor 104 is configured to generate an electronic visualization and control display of the generated electronic visualization to visually represent the unlabeled dataset of the unknown subject or other unknown subjects to distinguish the responders from the non-responders.

FIG. 3A is a diagram for identifying feature genes of a drug to obtain a score using a regression model, in accordance with an embodiment of the present disclosure. FIG. 3A is described in conjunction with elements from FIGS. 1A and 1B. With reference to FIG. 3A, there is shown a diagram 300A for identifying feature genes of a drug to obtain a score using the regression model 120 (of FIG. 1A), which may also be referred to as a feature selection regression model. Moreover, the processor 104 (of FIGS. 1A and 1B) is configured to identify the feature genes of a drug to obtain the score using the regression model 120 through a series of operations from 302-to-308.

At operation 302, the processor 104 is configured to perform initial data processing that includes three different operations 302A-to-302C. At operation 302A, the processor 104 is configured to receive input data, which may also be referred to as the first type of input of labeled pretreatment data 112 (of FIG. 1) of a plurality of subjects. In addition, at operation 302B, the processor 104 is configured to receive tumor volume change value, which may also be referred to as the second type of input of labeled post-treatment response data 114 (of FIG. 1) of the plurality of subjects for the one or more drugs. Moreover, at operation 302C, the processor 104 is configured to perform data augmentation, such as to generate the modified labeled training dataset 116. Furthermore, training an ensemble machine learning (ML) model 118 from the modified labeled training dataset 116. In addition, at operation 304, the processor 104 is configured to use the regression model 120 (of FIG. 1A), such as a tree-based (or a decision-tree based) regression model. In addition, at operation 306, a feature score is assigned to each gene in the modified labeled training dataset to identify one or more feature biomarkers of the one or more drugs. Finally, at operation 308, an output table is obtained, which is used to train the prediction model 126 (of FIG. 1A) based on the set of features extracted via the ensemble ML model 118.

FIG. 3B is a diagram for identifying feature genes of a drug to obtain a score using a classification model, in accordance with an embodiment of the present disclosure. FIG. 3B is described in conjunction with elements from FIGS. 1A and 1B. With reference to FIG. 3B, there is shown a diagram 300B for identifying feature genes of a drug to obtain a score using the classification model 122 (of FIG. 1A), which may also be referred to as a feature selection classification model. Moreover, the processor 104 (of FIGS. 1A and 1B) is configured to identify the feature genes of a drug to obtain the score using the classification model 122 through a series of operations from 302, 310-to-314.

At operation 302, the processor 104 is configured to perform initial data processing that includes three different operations 302A-to-302C. At operation 302A, the processor 104 is configured to receive input data, which may also be referred to as the first type of input of labeled pretreatment data 112 (of FIG. 1A) of a plurality of subjects. In addition, at operation 302B, the processor 104 is configured to receive tumor volume change value, which may also be referred to as the second type of input of labeled post-treatment response data 114 (of FIG. 1A) of the plurality of subjects for the one or more drugs. Moreover, at operation 302C, the processor 104 is configured to perform data augmentation, such as to generate a modified labeled training dataset 116 (of FIG. 1A). Furthermore, training an ensemble machine learning (ML) model 118 (of FIG. 1A) from the modified labeled training dataset 116 (of FIG. 1A). Thereafter, at operation 310, the processor 104 is configured to use tree-based (or decision-tree based) classification model (or the classification model 122 of FIG. 1A). In addition, at operation 312, a feature score is assigned to each gene in the modified labeled training dataset 116 (of FIG. 1A) to identify one or more feature biomarkers of the one or more drugs. Finally, at operation 314, an output table is obtained, which is used to train the prediction model 126 (of FIG. 1A) based on the set of features extracted via the ensemble ML model 118 (of FIG. 1A).

FIG. 4 is a diagram that represents a dendrogram to segregate responders and non-responders for a given drug, in accordance with an embodiment of the present disclosure. FIG. 4 is described in conjunction with elements from FIGS. 1A, 1B, 3A, and 3B. With reference to FIG. 4, there is shown a diagram 300B that represents a dendrogram to segregate responders and non-responders for a given drug.

In an implementation, the processor 104 (of FIG. 1) is configured to generate and control the display of the dendrogram to visually represent the unlabeled dataset of the unknown subject or other unknown subjects to distinguish the responders from the non-responders. Moreover, the dendrogram is used to get the visual clue about segregation of unknown patient samples from known responder or non-responder samples. In an implementation, the dendrogram of FIG. 4 is developed using top relevant features and using a visual representation of unlabelled data of the unknown subjects. In addition, the dendrogram can be prepared to get the perfect segregation of labels and to showcase the results, such as shown in FIG. 4 with a number of dots that are different for the responders, as compared to non-responders.

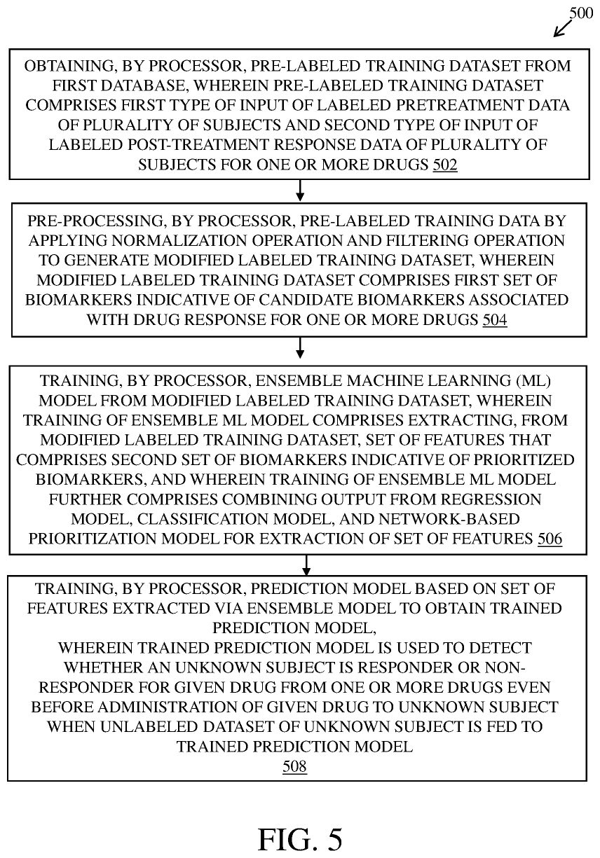

FIG. 5 is a flowchart of a method for an electronic evaluation of responders and non-responders for one or more drugs, in accordance with an embodiment of the present disclosure. FIG. 5 is described in conjunction with elements from FIGS. 1A, 1B, 3A, 3B and 4. With reference to FIG. 5, there is shown a flowchart of a method 500 for an electronic evaluation of responders and non-responders for one or more drugs. Moreover, the processor 104 (of FIGS. 1A and 1B) is configured to execute the method 500 that includes a series of steps from 502-to-508. The method 500 is used for an electronic evaluation of responders and non-responders for one or more drugs.

At step 502, the method 500 comprises, obtaining, by the processor 104, the pre-labeled training dataset 110 from the first database 108. In an implementation, the first database 108 is stored in the memory 106 of the server 102. In another implementation, the first database 108 is stored outside the memory 106. Moreover, the pre-labeled training dataset 110 includes the first type of input of labeled pretreatment data 112 of a plurality of subjects and the second type of input of labeled post-treatment response data 114 of the plurality of subjects for the one or more drugs. In an example, the first type of input of the labeled pretreatment data 112 corresponds to a genomics expression data of the plurality of subjects. In other words, upon receiving the first type of input of the labeled pretreatment data 112 of a disease, the processor 104 receives the second type of input of labeled post-treatment response data 114 related to one or more drugs of interest for which the plurality of subjects is to be screened to further gain mechanistic insights into the action of the one or more drugs.

At step 504, the method 500 comprises, pre-processing, by the processor 104, the pre-labeled training dataset 110 by applying a normalization operation and a filtering operation to generate the modified labeled training dataset 116. Moreover, the modified labeled training dataset 116 includes a first set of biomarkers indicative of candidate biomarkers associated with drug response for the one or more drugs. In an implementation, the normalization operation is used for selecting the second type of input from the pre-labeled training dataset 110 related to at least one drug from the one or more drugs from a list of response samples and non-response samples of the plurality of subjects. In an implementation, the filtering operation includes segregating and filtering out biomarkers that do not correlate with the drug response for the one or more drugs of a disease in the pre-labeled training dataset 110.

In an implementation, the normalization operation includes executing a Z-score log-normalization or other standard deviation-based log-normalization to reduce data noise in the pre-labeled training dataset. In an example, the processor 104 is configured to execute the Z-score log-normalization on the pre-labeled training dataset 110 (e.g., expression data) to generate the modified labeled training dataset 116. In another example, the processor 104 is configured to execute the standard deviation-based log-normalization, such as proprietary algorithms on the pre-labeled training dataset 110 (e.g., expression data) to generate the modified labeled training dataset 116.

At step 506, the method 500 comprises, training, by the processor 104, the ensemble machine learning (ML) model 118 from the modified labeled training dataset 116. Moreover. the training of the ensemble ML model 118 includes extracting, from the modified labeled training dataset 116, a set of features that includes a second set of biomarkers indicative of prioritized biomarkers. Further, the training of the ensemble ML model 118 further includes combining output from the regression model 120, the classification model 122, and the network-based prioritization model 124 for the extraction of the set of features.

In an implementation, the filtering operation includes segregating and filtering out biomarkers that do not correlate with the drug response for the one or more drugs of a disease in the pre-labeled training dataset. For example, the processor 104 is configured to analyze the drug response for the one or more drugs of the disease for a subject, such as a drug response associated with tumor volume change dataset along with the first type of input of labeled pretreatment data 112 for the same subject for which the information of responders/non-responder's values is related.

In an implementation, the method 500 further comprises assigning, by the processor 104, a first score to each gene in the modified labeled training dataset 116 to identify one or more feature biomarkers of the one or more drugs in the regression model 120, such as the regression model 120 is a decision-tree based regression model. In other words, the modified labeled training dataset 116 is used for identification as well as selection of the one or more feature biomarkers to train the decision-tree based regression model, such as each feature biomarkers from the second set of biomarkers later can be prioritized using the first score. In an example, the first score is used with a clustering model to label the importance genes based on covariance similarity.

In an implementation, the method 500 further comprises assigning, by the processor 104, a second score to each gene in the modified labeled training dataset 116 to identify one or more feature biomarkers of the one or more drugs in the classification model 122, such as the classification model 122 is a gradient boosting-based classification. In other words, the modified labeled training dataset 116 is used for identification as well as the selection of the one or more feature biomarkers to train the decision-tree based regression model, such as each feature biomarker from the second set of biomarkers later can be prioritized using the second score.

In an implementation, the method 500 further comprises computing, by the processor 104, a cumulative score in the network-based prioritization model 124 based on the first score and the second score of a corresponding gene to generate the set of features that includes the second set of biomarkers indicative of the prioritized biomarkers. Moreover, the network-based prioritization model 124 is a gene network-based gene prioritization model. For example, the processor 104 is configured to use the network-based prioritization model 124 through a network gene prioritization algorithm based on signalling pathway impact analysis (SPIA) methodologies to generate the cumulative score, which may also be referred to as a cumulative confidence score, a gene-perturbation score, gene-based impact score incorporating features of the first score and the second score. In addition, the cumulative score is used by the processor 104 to determine how much the corresponding gene in the modified labeled training dataset 116 is beneficial for the system 100A of genes across pathways.

At step 508, the method 500 comprises, training, by the processor 104, the prediction model 126 based on the set of features extracted via the ensemble ML model 118 to obtain a trained prediction model. In an example, the processor 104 is configured to apply data augmentation methodologies to generalize and train the prediction model 126, such as by building a modified support vector machine (SVM) based hyperparameters tuned prediction model to predict a target class. Moreover, the trained prediction model is used to detect whether an unknown subject is a responder or a non-responder for a given drug from the one or more drugs even before administration of the given drug to the unknown subject when an unlabeled dataset of the unknown subject is fed to the trained prediction model. Beneficially as compared to conventional approaches, the trained prediction model can be used to predict, if the unknown subject will benefit from the given drug or not, such as through an expression data for the set of genes for the unlabeled dataset of the unknown subject and even before administration of the given drug. In an implementation, the method 500 further comprises generating and controlling the display, by the processor, of an electronic visualization to visually represent the unlabeled dataset of the unknown subject or other unknown subjects to distinguish the responders from the non-responders.

The steps 502 to 508 are only illustrative and other alternatives can also be provided where one or more steps are added, one or more steps are removed, or one or more steps are provided in a different sequence without departing from the scope of the claims herein.

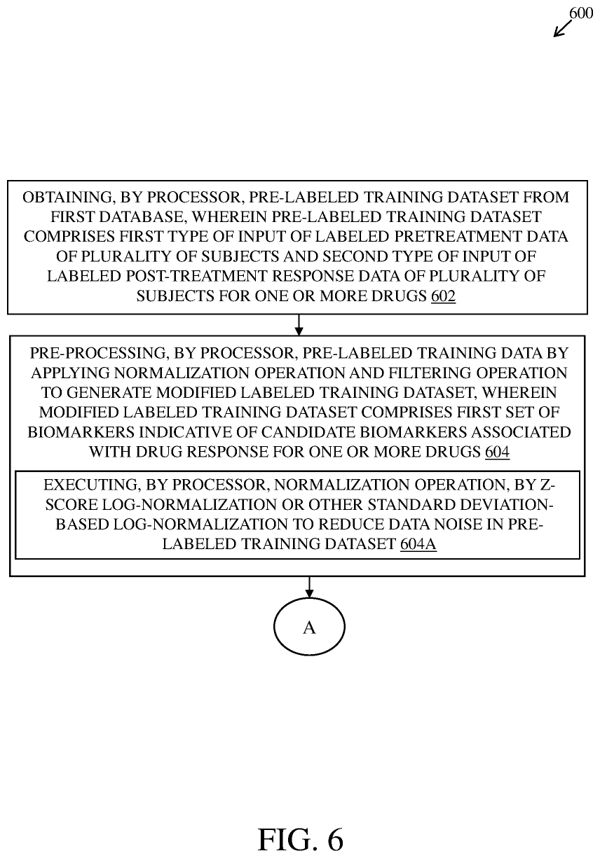

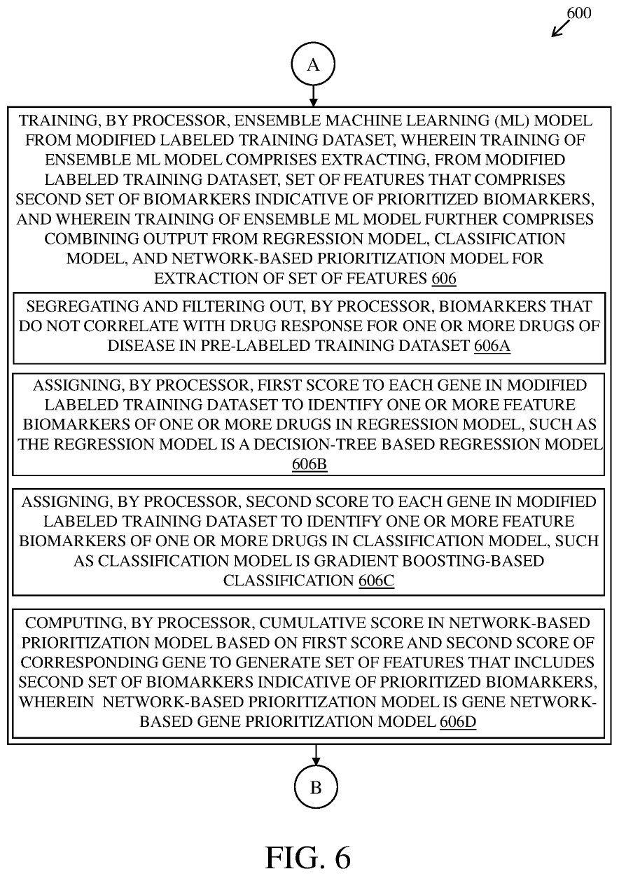

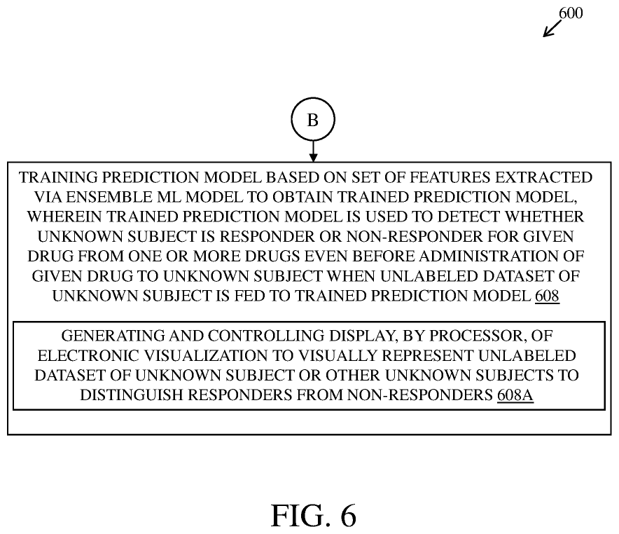

FIG. 6 is another flowchart of a method for an electronic evaluation of responders and non-responders for one or more drugs, in accordance with an embodiment of the present disclosure. FIG. 6 is described in conjunction with elements from FIGS. 1A, 1B, 3A, 3B and 4. With reference to FIG. 6, there is shown a flowchart of a method 600 for an electronic evaluation of responders and non-responders for one or more drugs. Moreover, the processor 104 (of FIGS. 1A and 1B) is configured to execute the method 600 that includes a series of steps from 602-to-608. The method 600 is used for an electronic evaluation of responders and non-responders for one or more drugs.

At step 602, the method 600 comprises, obtaining, by the processor 104, the pre-labeled training dataset 110 from the first database 108. Moreover, the pre-labeled training dataset 110 includes the first type of input of labeled pretreatment data 112 of a plurality of subjects and the second type of input of labeled post-treatment response data 114 of the plurality of subjects for the one or more drugs.

At step 604, the method 600 further comprises, pre-processing, by the processor 104, the pre-labeled training dataset 110 by applying a normalization operation and a filtering operation to generate the modified labeled training dataset 116. Moreover, the modified labeled training dataset 116 includes a first set of biomarkers indicative of candidate biomarkers associated with drug response for the one or more drugs.

At step 604A, the method 600 further comprises, executing, by the processor 104, the normalization operation, by a Z-score log-normalization or other standard deviation-based log-normalization to reduce data noise in the pre-labeled training dataset.

At step 606, the method 600 comprises, training, by the processor 104, the ensemble machine learning (ML) model 118 from the modified labeled training dataset 116. Moreover. the training of the ensemble ML model 118 includes extracting, from the modified labeled training dataset 116, a set of features that includes a second set of biomarkers indicative of prioritized biomarkers. Further, the training of the ensemble ML model 118 further includes combining output from the regression model 120, the classification model 122, and the network-based prioritization model 124 for the extraction of the set of features.

At step 606A, segregating and filtering out, by the processor 104, biomarkers that do not correlate with the drug response for the one or more drugs of a disease in the pre-labeled training dataset.

At step 606B, the method 600 further comprises, assigning, by the processor 104, a first score to each gene in the modified labeled training dataset 116 to identify one or more feature biomarkers of the one or more drugs in the regression model 120, such as the regression model 120 is a decision-tree based regression model.

At step 606C, the method 600 further comprises, assigning, by the processor 104, a second score to each gene in the modified labeled training dataset 116 to identify one or more feature biomarkers of the one or more drugs in the classification model 122, such as the classification model 122 is a gradient boosting-based classification.

At step 606D, the method 600 further comprises, computing, by the processor 104, a cumulative score in the network-based prioritization model 124 based on the first score and the second score of a corresponding gene to generate the set of features that includes the second set of biomarkers indicative of the prioritized biomarkers. Moreover, the network-based prioritization model 124 is a gene network-based gene prioritization model.

At step 608, the method 600 further comprises, training, by the processor 104, the prediction model 126 based on the set of features extracted via the ensemble ML model 118 to obtain a trained prediction model. Moreover, the trained prediction model is used to detect whether an unknown subject is a responder or a non-responder for a given drug from the one or more drugs even before administration of the given drug to the unknown subject when an unlabeled dataset of the unknown subject is fed to the trained prediction model.

At step 608A, the method 600 further comprises, generating and controlling display, by the processor 104, of an electronic visualization to visually represent the unlabeled dataset of the unknown subject or other unknown subjects to distinguish the responders from the non-responders.

The steps 602 to 608 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

1. A system for an electronic evaluation of responders and non-responders for one or more drugs, comprising:
a processor configured to:
obtain a pre-labeled training dataset from a first database, wherein the pre-labeled training dataset comprises a first type of input of labeled pretreatment data of a plurality of subjects and a second type of input of labeled post-treatment response data of the plurality of subjects for the one or more drugs;
pre-process the pre-labeled training data by applying a normalization operation and a filtering operation to generate a modified labeled training dataset, wherein the modified labeled training dataset comprises a first set of biomarkers indicative of candidate biomarkers associated with drug response for the one or more drugs;
training an ensemble machine learning (ML) model from the modified labeled training dataset, wherein training the ensemble ML model comprises extracting, from the modified labeled training dataset, a set of features that comprises a second set of biomarkers indicative of prioritized biomarkers, and wherein the ensemble ML model is configured to combine output from a regression model, a classification model, and a network-based prioritization model for the extraction of the set of features; and
train a prediction model based on the set of features extracted via the ensemble ML model to obtain a trained prediction model, wherein the trained prediction model is used to detect whether an unknown subject is a responder or a non-responder for a given drug from the one or more drugs even before administration of the given drug to the unknown subject when an unlabeled dataset of the unknown subject is fed to the trained prediction model.

2. The system according to claim 1, wherein the normalization operation comprises executing a Z-score log-normalization or other standard deviation-based log-normalization to reduce data noise in the pre-labeled training dataset.

3. The system according to claim 1, wherein the filtering operation comprises segregating and filtering out biomarkers that do not correlate with the drug response for the one or more drugs of a disease in the pre-labeled training dataset.

4. The system according to claim 1, wherein the regression model is a decision-tree based regression model in which a first score is assigned to each gene in the modified labeled training dataset to identify one or more feature biomarkers of the one or more drugs.

5. The system according to claim 4, wherein the classification model is a gradient boosting-based classification in which a second score is assigned to each gene in the modified labeled training dataset to identify one or more feature biomarkers of the one or more drugs.

6. The system according to claim 5, wherein the network-based prioritization model is a gene network-based gene prioritization model in which a cumulative score is computed based on the first score and the second score of a corresponding gene to generate the set of features that comprises the second set of biomarkers indicative of the prioritized biomarkers.

7. The system according to claim 1, wherein the processor is further configured to generate and control display of a dendrogram to visually represent the unlabeled dataset of the unknown subject or other unknown subjects to distinguish the responders from the non-responders.

8. A system for an electronic detection of responders and non-responders one or more drugs, comprising:
a processor configured to:
obtain an unlabeled dataset of an unknown subject from a second database;
pre-process the unlabeled dataset by applying a normalization operation to generate a modified unlabeled dataset;
extract one or more unlabeled features from the modified unlabeled dataset by executing a pre-trained ensemble machine learning (ML) model on the modified unlabeled dataset, wherein the pre-trained ensemble ML model is configured to combine output from a regression model, a classification model, and a network-based prioritization model to extract the one or more unlabeled features; and
detect whether the unknown subject is a responder or a non-responder for a given drug even before administration of the given drug to the unknown subject when the extracted one or more unlabeled features of the unknown subject is fed to a pre-trained prediction model, wherein the detection of the responder or the non-responder for the given drug comprises a concurrent screening of a group of biomarkers that collectively contribute to a drug response using the pre-trained prediction model.

9. The system according to claim 8, wherein the normalization operation comprises executing a Z-score log-normalization or other standard deviation-based log-normalization to reduce data noise.

10. The system according to claim 8, wherein the regression model is a decision-tree based regression model in which a first score is assigned to each gene in the modified unlabeled dataset.

11. The system according to claim 10, wherein the classification model is a gradient boosting-based classification in which a second score is assigned to each gene in the modified labeled training dataset.

12. The system according to claim 11, wherein the network-based prioritization model is a gene network-based gene prioritization model in which a cumulative score is computed based on the first score and the second score of a corresponding gene to generate the one or more unlabeled features.

13. The system according to claim 8, wherein the processor is further configured to generate an electronic visualization and control display of the generated electronic visualization to visually represent the unlabeled dataset of the unknown subject or other unknown subjects to distinguish the responders from the non-responders.

14. A method for an electronic evaluation of responders and non-responders for one or more drugs, comprising:
obtaining, by a processor, a pre-labeled training dataset from a first database, wherein the pre-labeled training dataset comprises a first type of input of labeled pretreatment data of a plurality of subjects and a second type of input of labeled post-treatment response data of the plurality of subjects for the one or more drugs;
pre-processing, by the processor, the pre-labeled training data by applying a normalization operation and a filtering operation to generate a modified labeled training dataset, wherein the modified labeled training dataset comprises a first set of biomarkers indicative of candidate biomarkers associated with drug response for the one or more drugs;
training, by the processor, an ensemble machine learning (ML) model from the modified labeled training dataset, wherein the training of the ensemble ML model comprises extracting, from the modified labeled training dataset, a set of features that comprises a second set of biomarkers indicative of prioritized biomarkers, and wherein the training of the ensemble ML model further comprises combining output from a regression model, a classification model, and a network-based prioritization model for the extraction of the set of features; and
training, by the processor, a prediction model based on the set of features extracted via the ensemble ML model to obtain a trained prediction model,
wherein the trained prediction model is used to detect whether an unknown subject is a responder or a non-responder for a given drug from the one or more drugs even before administration of the given drug to the unknown subject when an unlabeled dataset of the unknown subject is fed to the trained prediction model.

15. The method according to claim 14, wherein the normalization operation comprises executing a Z-score log-normalization or other standard deviation-based log-normalization to reduce data noise in the pre-labeled training dataset.

16. The method according to claim 14, wherein the filtering operation comprises segregating and filtering out biomarkers that do not correlate with the drug response for the one or more drugs of a disease in the pre-labeled training dataset.

17. The method according to claim 14, further comprising assigning, by the processor, a first score to each gene in the modified labeled training dataset to identify one or more feature biomarkers of the one or more drugs in the regression model, wherein the regression model is a decision-tree based regression model.

18. The method according to claim 17, further comprising assigning, by the processor, a second score to each gene in the modified labeled training dataset to identify one or more feature biomarkers of the one or more drugs in the classification model, wherein the classification model is a gradient boosting-based classification.

19. The method according to claim 17, further comprising computing, by the processor, a cumulative score in the network-based prioritization model based on the first score and the second score of a corresponding gene to generate the set of features that comprises the second set of biomarkers indicative of the prioritized biomarkers, wherein the network-based prioritization model is a gene network-based gene prioritization model.

20. The method according to claim 17, further comprising generating and controlling display, by the processor, of an electronic visualization to visually represent the unlabeled dataset of the unknown subject or other unknown subjects to distinguish the responders from the non-responders.
